(19) United States
(12) Patent Application Publication
(10) Pub. No.: US 2019/0159878 A1
(43) Pub. Date: May 30, 2019

(54) DUAL-PURPOSE CATHETER SYSTEM

(71) Applicant: TAYAL WASTY APPROACH LLC, Basking Ridge, NJ (US)

(72) Inventors: Najam Wasty, Basking Ridge, NJ (US); Raj Tayal, Warren, NJ (US)

(73) Assignee: TAYAL WASTY APPROACH LLC, Basking Ridge, NJ (US)

(21) Appl. No.: 16/198,214

(22) Filed: Nov. 21, 2018

Related U.S. Application Data
(60) Provisional application No. 62/590,403, filed on Nov. 24, 2017.

(51) Int. Cl. A61F 2/01; A61M 25/00; A61M 25/09; A61M 25/01

(52) U.S. Cl. CPC A61F 2/013; A61M 25/003; A61M 25/0041; A61F 2/2427; A61M 25/0108; A61M 25/0082; A61M 25/0905

(57) ABSTRACT

A dual-purpose catheter system for atheroemoblic stroke prevention in periprocedure Trans Catheter Aortic Valve replacement (TAVR) or Trans Catheter Mitral Valve Replacement (TMVR) in a patient by preventing atheroembolic debris (embolic plaque) from reaching the patient's brain and for contrast injection and filter immobilization/stabilization. The catheter system comprises: a delivery catheter, having two sections: a single-lumen Section A and a two-lumen Section B (a mother and a daughter lumen); a rectangular cerebrovascular filter housed in the single lumen of Section A; a removable 0.035″ J wire placed in section A and the mother lumen of section B; and an accompanying pigtail, catheter, which subsequent to filter deployment, is embedded in the single lumen of Section A and the mother lumen of Section B, traversing the under surface of the deployed filter. Methods for use and kits of the catheter system.

### TECHNICAL FIELD

This disclosure relates to the field of cardiac intervention. More specifically, this disclosure relates to a dual-purpose catheter system for use during Trans Catheter Aortic Valve replacement (TAVR) or Trans Catheter Mitral Valve Replacement (TMVR), with an inbuilt cerebrovascular protection filter to prevent or reduce stroke in a patient and an accompanying, separate, dedicated tensile pigtail catheter for contrast injection and filter immobilization/stabilization.

### BACKGROUND

Trans Catheter Aortic Valve replacement (TAVR) and Trans Catheter Mitral Valve Replacement (TMVR) are two new and revolutionary procedures used to repair or replace heart valves. The incidence, however, of periprocedure stroke is unacceptably high, as dislodged small plaques, mostly from the diseased valves, travel up to the brain via blood vessels feeding the brain. Thus, periprocedure embolic cerebrovascular accidents remain a major concern, with rates of clinically apparent, disabling strokes approximating 2-5% in TAVR and as high as 5-7% in TMVR procedures.

Devices with filters have been developed to reduce these risks, however only one is commercially available in the United States, and each has a myriad of clinical drawbacks. Initial studies have shown these filters to contain significant atherosclerotic debris post procedurally in more than 87% of cases.

### SUMMARY

This disclosure provides a dual-purpose catheter system for atheroembolic stroke prevention in periprocedure Trans Catheter Aortic Valve replacement (TAVR) or Trans Catheter Mitral Valve Replacement (TMVR) in a patient by preventing atheroembolic debris (embolic plaque) from reaching the patient's brain and for intraprocedural contrast injection and filter immobilization/stabilization. The dual-purpose catheter system comprises two catheters, a delivery catheter and an angled single-lumen dedicated tensile pigtail catheter.

In certain embodiments, the delivery catheter is 7 French in size and approximately 100 cm long, having a front end adapted to be placed in the aortic arch of the patient, covering the brachiocephalic artery takeoff, the left common carotid artery takeoff, and left subclavian artery takeoff, and a rear end. The delivery catheter is adapted to be inserted into a blood vessel in the leg of a patient percutaneously and then advanced to the aortic arch. When inserted, part of the rear end of the delivery catheter hangs outside the blood vessel and thus the body of the patient. In certain embodiments, the delivery catheter comprises two sections, a single-lumen section A that is approximately 12 cm of the front end of the delivery catheter and a double-lumen section B that constitutes the rest of the delivery catheter. The two lumens of section B are a mother lumen that is continuous with the single lumen of section A and a daughter lumen, which is a 0.018″ lumen embedded within the mother lumen along a plane traversing the entire length of section B of the delivery catheter.

In certain embodiments, a rectangular cerebrovascular filter is housed in the single lumen of section A of the delivery catheter. In certain embodiments, the filter is attached firmly to a 0.018″ wire running in the daughter lumen of the delivery catheter. The filter is made of suitable materials with a pore size suitable to filter debris and allow passage of blood. The filter is either in a folded, undeployed position inside the single lumen of section A when the delivery catheter is not retracted, or in an unfolded, deployed position when the delivery catheter is retracted. In certain embodiments, the filter has a strong tensile memory for an outward convexity so once deployed it firmly hugs the inner surface of the aortic arch of the patient. In certain embodiments, the filter is a thin collapsible mesh 2 to 4 cm wide and approximately 12 cm long. In certain embodiments, a firm but flexible 0.018″ wire is attached to the outer convex surface of the filter (on the “dorsum” of the filter) in section A of the delivery catheter. In certain embodiments, the 0.018″ wire runs along the entire length of the single lumen of section A and the daughter lumen of section B. In certain embodiments, the 0.018″ wire is approximately 120 cm in length with the front 12 cm attached to the filter in the single lumen of section A.

In certain embodiments, a removable and re-insertable 0.035″ J wire is placed in the mother lumen of section B and the single lumen of section A of the delivery catheter; the J wire thus runs along the entire length of the delivery catheter. In certain embodiments, the 0.035″ J wire is a moderately stiff approximately 260 cm long wire loosely embedded in the single lumen of section A and the mother lumen of section B of the delivery catheter such that the filter is given space to be safely housed and deployed without entangling the 0.035″ J wire. In certain embodiments, approximately 30 cm of the 0.035″ J wire is adapted to jut beyond the front end of the delivery catheter. In certain embodiments, a portion of the 0.035″ J wire is adapted so as to hang outside of the patient's blood vessel.

The system comprises a separate single-lumen dedicated angled pigtail catheter. In certain embodiments, the single-lumen dedicated angled pigtail catheter is 5 F. The pigtail catheter comprises side holes and features composite construction, lending it radial tensile strength. The delivery catheter is retractable, to deploy the filter. The pigtail catheter is adapted to be inserted from the rear of this delivery catheter over the 0.035″ J wire traversing the mother lumen of section B, thus occupying the single lumen of section A. The single lumen of section A, in the case when the filter is in the deployed position, now has enough space to house the 5 F single-lumen dedicated angled pigtail catheter.

The delivery catheter is retractable. And by retracting it (in the retracted position), in certain embodiments, by about 12 cm in the front, the filter is deployed; and in further embodiments, the pigtail catheter is inserted into the rear end of the delivery catheter, over the 0.035″ J wire traversing the mother lumen of section B, thus occupying the single lumen of section A. The single lumen of section A, sitting underneath the filter. In certain embodiments, the front most about 6 cm of the delivery catheter has extra inbuilt recoil. In certain embodiments, the 0.035″ wire is an introducer and is the wire on which the delivery catheter is railed into the body for filter and pigtail catheter delivery (once the filter has been deployed).

In other aspects, this disclosure provides a method of preventing or reducing atheroembolic debris from reaching the brain during heart valve repair or replacement in a patient in need of heart valve repair or replacement.

In certain embodiments, the method comprises: inserting a dual-purpose delivery catheter disclosed herein over the 0.035″ wire into a blood vessel in the leg of the patient and advancing the delivery catheter to the aortic arch of the patient, the filter in the delivery catheter is not yet deployed; telescopically retracting the delivery catheter by about 12 cm over the 0.018″ wire and the 0.035″ J wire, while holding the 018″ wire and the 0.035″ J wire with one hand (in certain embodiments, the right hand) thus deploying the filter; the pigtail catheter is inserted into the delivery catheter into the mother lumen of section B and through the single lumen of Section A over the 0.035″ J wire, the pigtail catheter traverses the entire mother lumen of section B and the single lumen of section A and hangs out the front end of the delivery catheter, in the ascending aorta, closely hugging the under surface of the deployed filter. The 0.035″ J wire is finally removed (withdrawn) from the delivery catheter. The pigtail catheter may be connected to a manifold. The filter covers the right brachiocephalic artery takeoff, the left common carotid artery takeoff, and the left subclavian artery takeoff snugly to prevent or reduce atheroembolic debris (embolic plaque) from going up to the brain and the pigtail catheter is used for contrast injection and filter immobilization/stabilization.

Numerous other aspects and embodiments are provided in accordance with these and other aspects of the invention. Other features and aspects of the present invention will become more fully apparent from the following detailed description and the appended claims.

### BRIEF DESCRIPTION OF THE DRAWINGS

FIG. 1A shows a left anterior oblique view of the aortic arch greater curvature and FIG. 1B shows an antero-posterior cranial view of the aortic arch greater curvature.

FIG. 2 shows a view of the pigtail catheter hugging the greater curvature of the aortic arch, its tip resting along the aortic root such that pushing the pigtail catheter into aortic cusp drives the filter into the greater curvature of the aortic arch.

FIG. 3 shows a composite tensile pigtail catheter with built in recoil generating centrifugal forces (arrows) when bent along the greater curvature of the aortic arch.

FIG. 4 shows a view of the greater curvature of the aortic arch and the centrifugal forces (arrows) generated by bending a catheter with recoil along it.

FIG. 5 shows a view of the aortic arch and the centrifugal forces generated by bending a catheter with recoil along the greater curvature of the aortic arch.

FIG. 6 shows a view of a type I aortic arch.

FIG. 7 shows a view of a type III aortic arch and its effect on enhancing the centrifugal forces (arrows) generated by the “hairpin effect” secondary to the increased angularity of the type III aortic arch.

FIG. 8 shows a view of a delivery catheter in the aortic arch, with the filter in the undeployed position, which has been delivered over the 0.035″ J wire.

FIG. 9 shows a view of a delivery catheter in the aortic arch, with the filter in the deployed position. Note that the 0.035″ J wire has been withdrawn after the insertion of the angled pigtail catheter, which now sits in an aortic cusp.

FIG. 10 shows a view of an embodiment of a delivery catheter.

FIG. 11 shows a view of an embodiment of the filter and other parts of the filter.

FIG. 12 shows a view of an embodiment of the 0.035″ J wire and other parts of the delivery catheter, including sponge plug at the front of the dual-purpose delivery catheter.

### DETAILED DESCRIPTION

As used herein, the word “a” or “plurality” before a noun represents one or more of the noun.

As used herein, the terms “subject” and “patient” are used interchangeably. A patient or a subject can be a human patient or a human subject.

As used herein, the terms “atheroembolic debris,” “embolic plaque,” “plaque,” “emboli,” and the like, are used interchangeably.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Heart Valve Repair or Replacement: TAVR and TMVR

Trans Catheter Aortic Valve Replacement (TAVR) and Trans Catheter Mitral Valve replacement (TMVR) are two new and revolutionary procedures used to repair or replace the aortic or mitral valve respectively in patients in need of a such repair or replacement, patients who suffer from aortic valve or mitral valve stenosis, or mitral valve regurgitation.

Aortic stenosis is a narrowing of the aortic valve opening. Aortic stenosis restricts the blood flow from the left ventricle to the aorta and may also affect the pressure in the left atrium. Mitral stenosis is a valvular heart disease characterized by the narrowing of the orifice of the mitral valve of the heart.

Mitral valve regurgitation is a condition in which the heart's mitral valve doesn't close tightly, which allows blood to flow backward in the heart.

TAVR may be performed by inserting a catheter through the femoral artery (large artery in the groin), called the transfemoral approach, which does not require a surgical incision in the chest; or it may be performed using a minimally invasive surgical approach with a small incision in the chest and inserting the catheter through the tip of the left ventricle (the apex), which is known as the transapical approach. TMVR may be performed by the transseptal route.

The incidence, however, of stroke during and after TMVR and TAVR is unacceptably high, as dislodged atheroembolic debris (in the form of small plaques) during these procedures, mostly from the damaged valves, travelling up to the brain via the three branches of the aortic arch, the brachiocephalic artery (which in turn divides into the right common carotid artery and the right subclavian artery, which gives rise the right vertebral artery, supplying blood to the posterior part of the brain), the left common carotid artery, and the left subclavian artery, which gives rise the left vertebral artery, also supplying blood to the posterior part of the brain. Preventing these small plaques from going up these arteries and reaching the brain is essential to prevent or reduce the incidence and/or severity of stroke in the patient during and after these cardiac interventions.

When performing such cardiac interventions, variation in aortic arch may need to be considered. There are 3 types of aortic arch, as assessed by the relationship of the brachiocephalic vessels takeoff. Type 1 arch represents a relatively level aortic arch. Angulation of the aortic arch is classified as type 2 or type 3, depending on the degree of angulation.

Currently Available Filters

Filter systems with limitations are currently available for preventing plaques from going up to the brain. They are, however, too cumbersome to deploy, require a radial approach, and/or can themselves cause stroke. Also, each of these filters is separate from the pigtail catheter, thus requiring a separate arterial access site for the pigtail catheter.

These filters may cause stroke because they scrape by the brain arteries as they are passed on to the aortic arch, or because one of their anchoring prongs sits in the right brachiocephalic artery, thus potentially scraping atheroma.

The design of these filters can also prevent full coverage of all the major brain arteries, such as missing the Left Vertebral Artery. In one case, one of the anchoring prongs of the device straddles across the lumen of the aorta potentially creating an impediment to and risk of entanglement with the advancing TAVR Catheter. Or their design features an inbuilt concavity of the deployed filter that prevents the filter from following the natural circumferential convexity of the aorta arch leaving a gap between the deployed filter and the major aortic arch branches/MAAB takeoff. thereby providing less than a perfect seal and potential for atheroemboli to “sneak by” to the brain causing stroke.

Dual-Purpose Catheters

In certain aspects, a dual-purpose catheter system is provided for atheroembolic stroke prevention in periprocedure Trans Catheter Aortic Valve replacement (TAVR) or Trans Catheter Mitral Valve Replacement (TMVR) in a patient, with a cerebrovascular protection filter to prevent or reduce stroke in a patient and a separate, dedicated tensile pigtail catheter for contrast injection and filter immobilization/stabilization. The dual-purpose catheter system comprises two catheters, a delivery catheter and a dedicated tensile pigtail catheter.

The delivery catheter may be any suitable size. In certain embodiments, the delivery catheter is 7 French in size and approximately 100 cm long. The front end (also referred to herein as front and the like) of the delivery catheter is adapted to be inserted into a blood vessel and advanced to the aortic arch of a patient. The rear end (also referred to herein as rear, back, back end, and the like) of the delivery catheter is adapted to lag outside the blood vessel and thus outside of the patient's body. When the delivery catheter is inside the blood vessel in the leg of a patient and advances to the target position in the aortic arch, part of the rear of the delivery catheter is adapted to hang outside of the blood vessel in the leg and thus the body of the patient.

The delivery catheter comprises two sections: a single-lumen section A that is, in certain embodiments, approximately 12 cm of the front end of the delivery catheter, and a double-lumen section B that is the rest of the delivery catheter. The two lumens of section B are a mother lumen that is continuous with the single lumen of Section A and a daughter lumen located along the top circumference of the mother lumen. In certain embodiments, the daughter lumen is 0.018″. The daughter lumen is embedded within the mother lumen along a plane traversing the entire length of Section B.

A cerebrovascular filter (which in certain embodiments is rectangular) is housed in the single lumen of Section A of the delivery catheter. The filter is either in a folded, undeployed position inside the single lumen of section A when the delivery catheter is not retracted or in an unfolded, deployed position when the delivery catheter is retracted. In certain embodiments, the filter has a strong tensile memory for an outward convexity to hug the inner surface of the aortic arch of the patient. The filter may be a thin collapsible mesh (in certain embodiments, the filter is 2 to 4 cm wide) (and in certain embodiments, is approximately 12 cm long), firmly attached to a firm but flexible wire that may be a 0.018″ wire housed in the daughter lumen, which in certain embodiments is 0.018″ (this wire may be the same dimension as the daughter lumen), running along the entire length of the daughter lumen of Section B and firmly attached to the dorsum of the folded filter in the single lumen of section A. The 0.018″ wire may be approximately 120 cm in length with the front 12 cm in the single lumen of section A attached to the dorsum of the filter.

A removable (and re-insertable) J wire (in certain embodiments, it is 0.035″) is housed in the mother lumen of section B and the single lumen of section A of the delivery catheter. The J wire runs along the entire length of the delivery catheter and is a moderately stiff (in certain embodiments, approximately 240 cm-260 cm) long wire loosely housed in the single lumen of section A and the mother lumen of section B of the delivery catheter. There is enough space in Section A for the filter to be housed without entangling the J wire. A certain length (in certain embodiments, approximately 30 cm) of the 0.035″ J wire may protrude beyond the front end of the delivery catheter and/or a certain length of the J wire may hang outside the rear of the delivery catheter.

The system comprises a separate angled single-lumen dedicated pigtail catheter. In certain embodiments, the pigtail catheter is 5 F. The pigtail catheter has side holes and features composite construction, lending it radial tensile strength. It is adapted to be inserted into the back (rear) of the delivery catheter over the 0.035″ J wire (once the delivery catheter is retracted—in the retracted position—and the filter is deployed and delivered—in the deployed position) into the mother lumen of Section B and becomes housed within the single lumen of section A and the mother lumen of section B, once it is introduced after filter deployment. The pigtail catheter is for contrast injection and filter immobilization/stabilization.

The delivery catheter is designed to be retractable by the length of section A (in certain embodiments, about 12 cm) from the front end toward the back end to allow the filter to deploy upon retraction of the catheter, also allowing the angled 5 F pigtail catheter to now advance over the 0.035″ wire after the filter has unfolded and deployed and the pigtail catheter inserted into the mother lumen of section B and the single lumen of section A of the delivery catheter. When the delivery catheter is not retracted, the filter is folded and not deployed, stored in the lumen of Section A; when the delivery catheter is retracted, the filter is deployed.

The J wire is an introducer and in certain embodiments comprises an introducer rail. The J wire is the wire on which the delivery catheter is railed into the body for filter and pigtail catheter delivery.

In certain embodiments, the delivery catheter is made with composite construction and about first 6 cm at the front end of Section A of the delivery catheter comprises spring-like flexible ribbing with extra centrifugal recoil and tensile strength that transmits to the deployed filter additional close snuggle against the major aortic arch branches (MAAB) takeoff, especially in patients with type 2 arch or type 3 arch. Most elderly patients with critical aortic stenosis have type 2 arch or type 3 arch.

This catheter system is suitable for use for all 3 types of aortic arch.

In certain embodiments, two radio opaque markers, one placed at the front of section B of the delivery catheter and one embedded along the very same plane at the front of section A of the delivery catheter, are used to confirm the orientation of the daughter lumen. The two markers are placed on the superior aspect of the dual-purpose catheter along the plane of the 0.018″ daughter lumen housing the 0.018″ wire to which the filter is attached. The two markers facilitate proper alignment and deployment by rotating the delivery catheter to ensure proper filter deployment against the greater curvature of the MAAB takeoff. In certain embodiments, the delivery catheter has two proximal flush ports to facilitate air removal from both mother and daughter lumen. In certain embodiments, the delivery catheter further comprises rubber seals placed at the back end of the delivery catheter, guarding both the 0.018″ and 0.035″ wire portals against air entry. In certain embodiments, the delivery catheter comprises both 0.018″ and 0.035″ wire portals at the back and 0.035″ wire portal in the front. In certain embodiments, a clip-like removable sponge cap guards the front end of the delivery catheter, allowing saline flush to exit; but once soaked, the sponge cap prevents air from re-entering the catheter by providing a seal against air entry. This sponge cap is removed just prior to introducing the catheter in the body to reduce the potential for any air embolism.

In certain embodiments, the filter is made of materials that allow for blood to pass through but impedes passage of the larger atheroembolic debris. In certain embodiments, the filter, when deployed, has a strong tensile memory for an outward convexity designed to hug the inner surface of the aortic arch.

The filter, once deployed, hugs the inner surface of the aortic arch and in addition is immobilized (held in place) by the pigtail catheter sitting against the aortic cusp and due to the natural cresting of the MAABs along the greater curvature of the aortic arch. The filter hugs the takeoff of the MAAB from the greater curvature of the aortic arch.

In certain embodiments, a sponge clip 4 mm in length and 7 F in diameter is wrapped around the 0.035″ wire to cap the front end of the delivery sheath to prevent air entry from this end.

In certain embodiments, the angled pigtail catheter is a single lumen 5 F angled pigtail catheter with side holes featuring composite construction lending it extra tensile centrifugal recoil. The pigtail catheter is for contrast injections, pressure measurements, and adjunctive filter immobilization/stabilization.

The pigtail catheter, once inserted into the mother lumen of section B and the single lumen of section A of the delivery catheter (over the 0.035″ wire, which is ultimately removed) after filter deployment, can freely glide back and forth during the entirety of the TAVR or TMVR procedure for the requisite frequent contrast injections. Added centrifugal forces generated by the pigtail catheter bending along the greater curvature of the aortic arch as it runs along the undersurface of the deployed filter, further ensuring enhanced snuggling of the filter across the major aortic arch branch (MAAB) takeoff.

The angled pigtail catheter also provides another simple mechanism for immovably fixating the deployed filter against the MAAB takeoff by simply pushing the pigtail catheter into the aortic cusp during TAVR/TMVR procedures. This simple maneuver totally obviates the need for any cumbersome anchoring prongs.

In other aspects, this disclosure provides a method of preventing or reducing atheroembolic debris from reaching the brain during heart valve repair or replacement in a patient in need of heart valve repair or replacement, comprising: inserting a delivery catheter disclosed herein into a blood vessel in the leg of the patient and advancing the dual-purpose catheter to the aortic arch of the patient (front end first) by the 0.035″ J wire looping in on an aortic cusp (usually the right), the filter on the dual-purpose delivery catheter is not yet deployed; Telescopically retracting the delivery catheter over the 0.018″ wire and the 0.035″ wire, deploying the filter, which is immobilized against the major aortic arch branch (MAAB) takeoff; inserting the pigtail catheter disclosed herein into the mother lumen of section B and the single lumen of section A of the delivery catheter, wherein the pigtail catheter traverses the entire delivery catheter and sits underneath the filter in the aortic arch; Wherein the filter covers the right brachiocephalic artery takeoff, the left common carotid artery takeoff, and the left subclavian artery takeoff snugly to prevent or reduce atheroembolic debris (embolic plaque) from going up to the brain of the patient during TMVR or TAVR.

In further embodiments, the method comprises removing (withdrawing) the 0.035″ J wire, leaving behind the pigtail catheter in the ascending aorta for contrast injections, pressure measurements and filter immobilization.

In further embodiments, the method further comprises withdrawing the pigtail catheter over the 0.035″ J wire (which in these embodiments is not withdrawn or is re-inserted into the delivery catheter) and removing the pigtail catheter from the body. As the pigtail catheter is being withdrawn the 035″ J wire is simultaneously advanced all the way to the aortic root and jammed/looped against an aortic cusp to keep the filter immobilized. Holding the dual-purpose delivery catheter and the 0.035′ wire firmly in one hand the 0.018″ wire is now gently withdrawn until the filter attached to it is fully re-encased in the single lumen section A of the dual-purpose delivery catheter. Once the filter is thus fully re-sheathed the 0.018″ wire and the dual-purpose catheter now completely housing the retrieved filter are held firmly in one hand and the 0.035″ J wire is pinned down to the table by the other hand to keep it immobilized as the dual-purpose catheter housing the filter is “walked out” of the body of the patient over the 0.035″ J wire. Once the dual-purpose catheter containing the filter is completely out of the body the 0.035″ wire can also be withdrawn fully and removed from the body of the patient. In certain further embodiments, the cardiac intervention procedure has been completed before these steps are performed.

The methods disclosed require only a single blood vessel access point in the patient, as both the filter and the pigtail catheter are delivered by the delivery catheter.

Where specific size, shape, or dimension is given herein, the size, shape, or dimension may be changed as needed.

The disclosed method is suitable for use for all 3 types of aortic arch.

This disclosure also provides a kit comprising the disclosed catheter system for use in a disclosed method. The kit may further comprise instructions and/or warning for use.

The design of the disclosed catheter system is based on simple rules of physics, namely bent catheters with strong tensile properties generates strong centrifugal forces and the simple anatomical fact that all MAAB take off along the superior most and the center most aspect of the greater curvature of the aortic arch.

The disclosed delivery catheter may be inside a kit along with the disclosed pigtail catheter; it may be outside of a kit but not inserted into a patient's blood vessel; or it may be inserted into a patient's blood vessel and placed in the aortic arch. While inside a patient's body, the delivery catheter be unretracted and thus the filter is not deployed, or the delivery catheter is retracted and the filter is deployed. Once the filter is deployed, the pigtail catheter may be inserted into the blood vessel of the patient and into the delivery catheter over the 0.035″ J wire. Once the pigtail catheter is in place, the 0.035″ J wire may be withdrawn.

The disclosed dual-purpose catheter's design is based on the following:

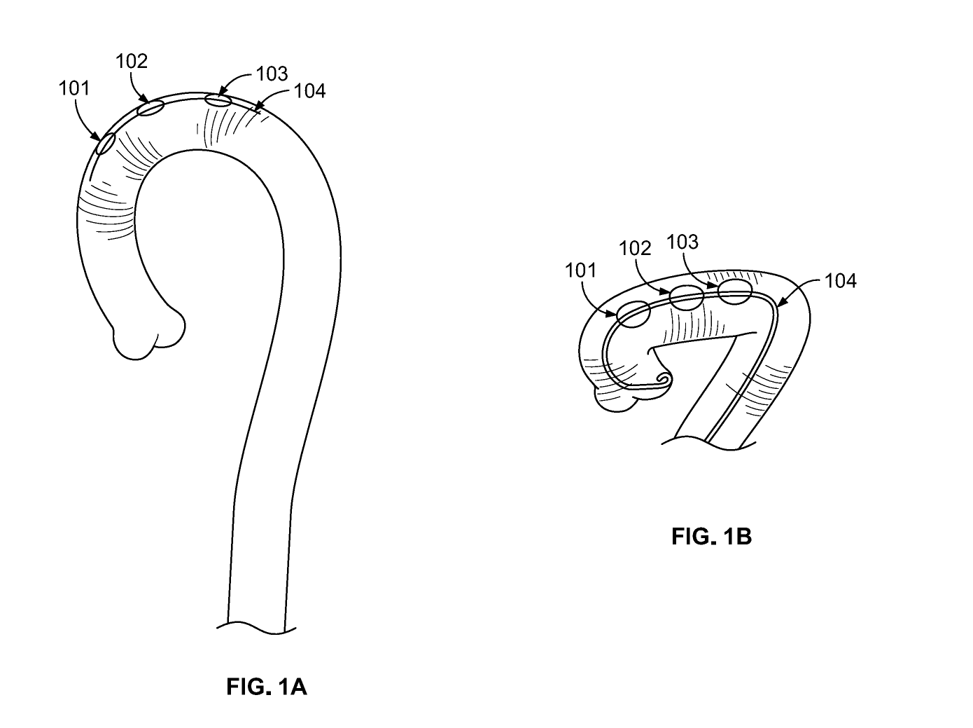

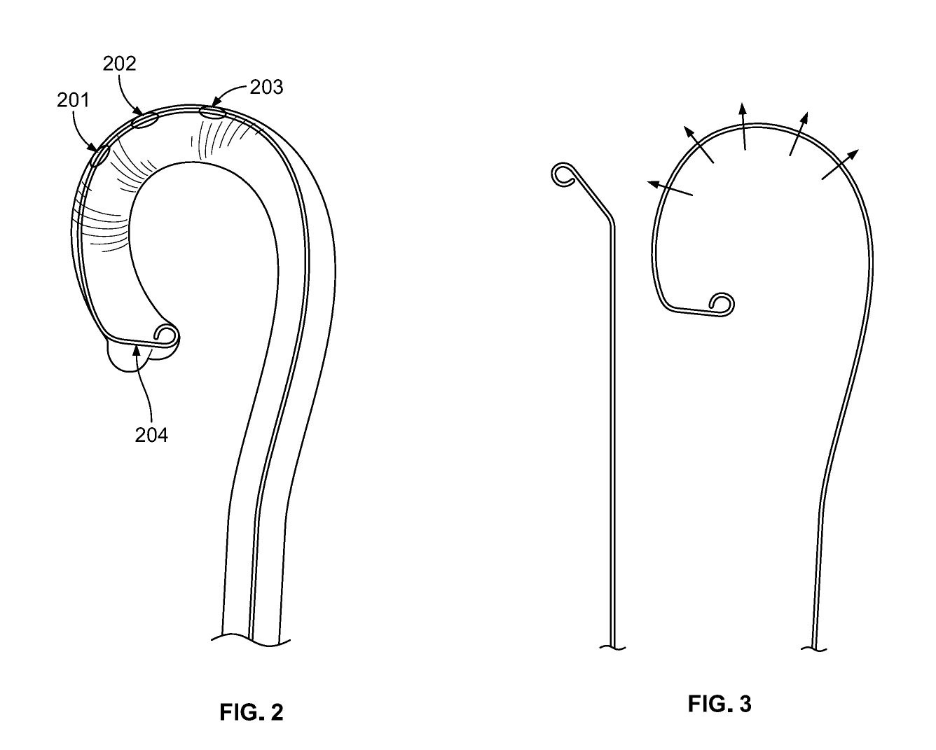

1) The MAABs are crested along the superior most and center most aspect of the greater curvature of the aortic arch. See FIG. 1 and FIG. 2.

FIG. 1A (left anterior oblique view) and FIG. 1B (antero-posterior cranial view) shows the aortic arch greater curvature, which is the superior most and central most aspect of the aortic arch. Shown are the right brachiocephalic artery 101; the left common carotid artery 102; and the left subclavian artery 103. FIG. 1B (antero-posterior cranial view) shows the aortic arch greater curvature, which is the superior most and central most aspect of the aortic arch. Shown are the right brachiocephalic artery 101; the left common carotid artery 102; and the left subclavian artery 103, and placement of an angled tensile pigtail catheter 104.

FIG. 2 shows the aortic arch greater curvature, superior most and central most aspect of the aortic arch. Shown are the right brachiocephalic artery 201; the left common carotid artery 102; and the left common carotid artery 203. It also shows that an angled tensile pigtail catheter 204 (indeed all catheters) advancing or retreating hugs the aortic crest and the MAAB takeoff from the greater curvature.

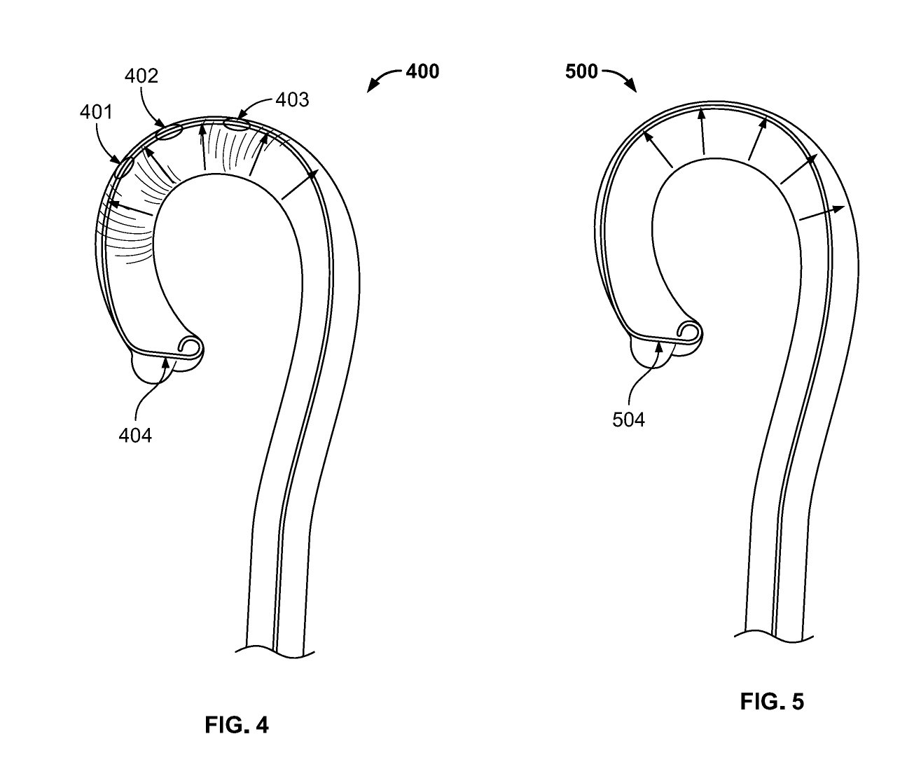

2) When approaching from the leg ALL advancing and retreating catheters following simple rules of physics must hug the greater curvature of the aortic arch; the greater the inbuilt catheter tensile recoil the greater the centrifugal forces generated and the tighter this hugging effect. See FIG. 3 and FIG. 4.

FIG. 3 shows the centrifugal force (arrows) generated by a pig tail catheter with inbuilt recoil. On the left is the pig tail catheter that is straight, as it comes out of the box (container or kit). On the right is the pig tail catheter that is bent along the aortic arch, generating centrifugal force (arrows).

FIG. 4 shows the centrifugal force (arrows) generated by bending a catheter with recoil. The catheter pushes against the MAAB takeoff. Shown are the right brachiocephalic artery 401; the left common carotid artery 402; and the left subclavian artery 403.

3) Because most elderly patients with critical aortic stenosis being evaluated for TAVR have a Type 2 or 3 aortic arch, there is an increased “hair pinning” effect on all tensile catheters traversing along these aortic arches generating even stronger centrifugal forces causing even tighter catheter snuggle against the MAAB takeoff from the greater curvature of the Aortic arch. See FIG. 5 and FIG. 7.

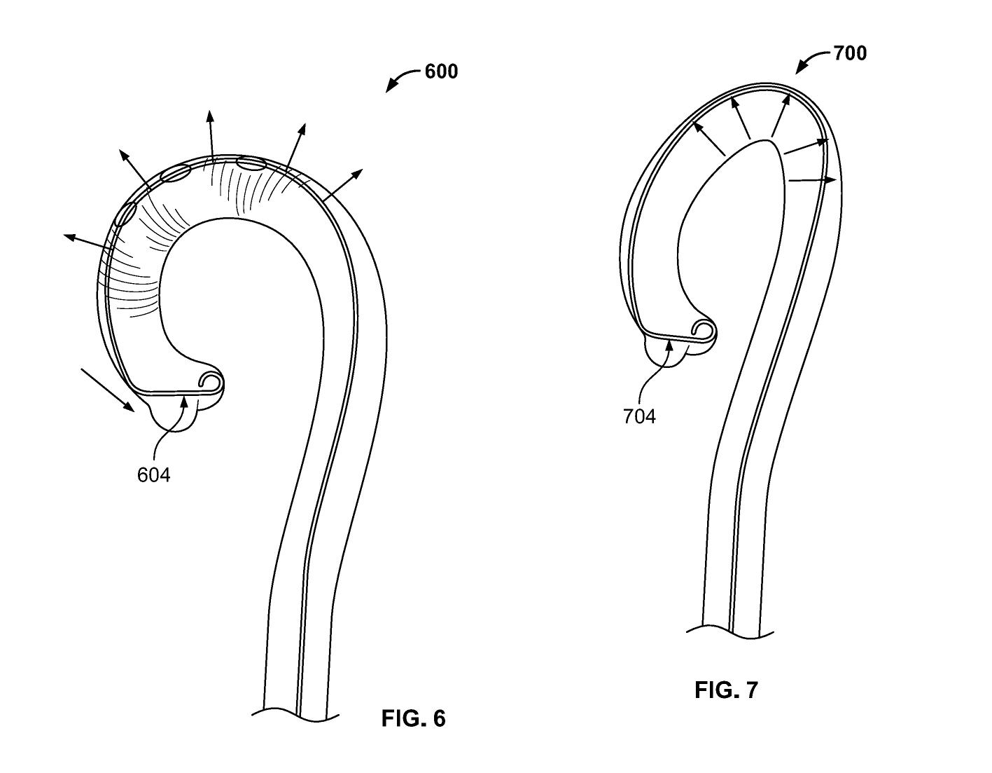

FIG. 6 shows a type I aortic arch 600. An angled tensile pigtail catheter 604 is bent along the aortic arch, generating centrifugal force (arrows).

FIG. 7 shows a type III aortic arch 700. An angled tensile pigtail catheter 704 is bent along the aortic arch, generating centrifugal force (arrows). The hairpin effect of type III aortic arch in critical aortic stenosis subjects is shown. This effect creates enhanced centrifugal force when the pigtail catheter, the filter, and the dual-purpose catheter are in proper position, with more recoil against and hugging of MAAB takeoff.

4) The dedicated tensile pigtail catheter not only generates additional centrifugal forces as it traverses along the under surface of the deployed filter, pushing the deployed filter against the MAAB takeoff but can also be used to totally immobilize the deployed filter along the greater curvature of the Aortic Arch by pushing the distal end of the Pigtail Catheter against an aortic cusp in TAVR and TMVR procedures especially during the phases of the procedure when filter movement could potentially occur as when the bulky TransCatheter Aortic valve delivery system is being advanced across the Aortic Arch during TAVR. See FIG. 9. Pigtail catheter in diagram sits against the aortic cusp.

FIG. 5 shows the centrifugal force (up arrows) generated by an angled tensile pigtail catheter 504 bent in the aortic arch. The effect of the centrifugal force can be enhanced by jamming the pigtail catheter into/against the aortic roof; more the pushdown (down arrow) more the centrifugal force (up arrows) (action and reaction are equal and opposite). On the left is the pig tail catheter that is straight as it comes out of the box. On the right is the pigtail catheter that is bent along the aortic arch.

The disclosed catheter system is suitable for use in patients with any of the three types of aortic arch.

The disclosed catheter system may be produced by suitable methods known in the art and suitable materials known in the art. A person of ordinary skill in the art, armed with this disclosure, is able to make the disclosed catheter system, using suitable components and materials known in the art.

The delivery catheter or the pigtail catheter may be made of any suitable material. Either catheter may be made by, for example, a range of polymers, including, without limitation, silicone rubber, nylon, polyurethane, polyethylene terephthalate (PET), latex, silicon elastic, and thermoplastic elastomers.

The filter may be made of any suitable material, such as, for example, stainless steel, titanium. The filter may comprise polyurethane membrane (or another suitable polymeric material) with pores.

The 0.018″ wire and the 0.035″ wire may be made of any suitable material, such as stainless steel or Nitinol.

### REFERENCE TO SOME OF THE DRAWINGS

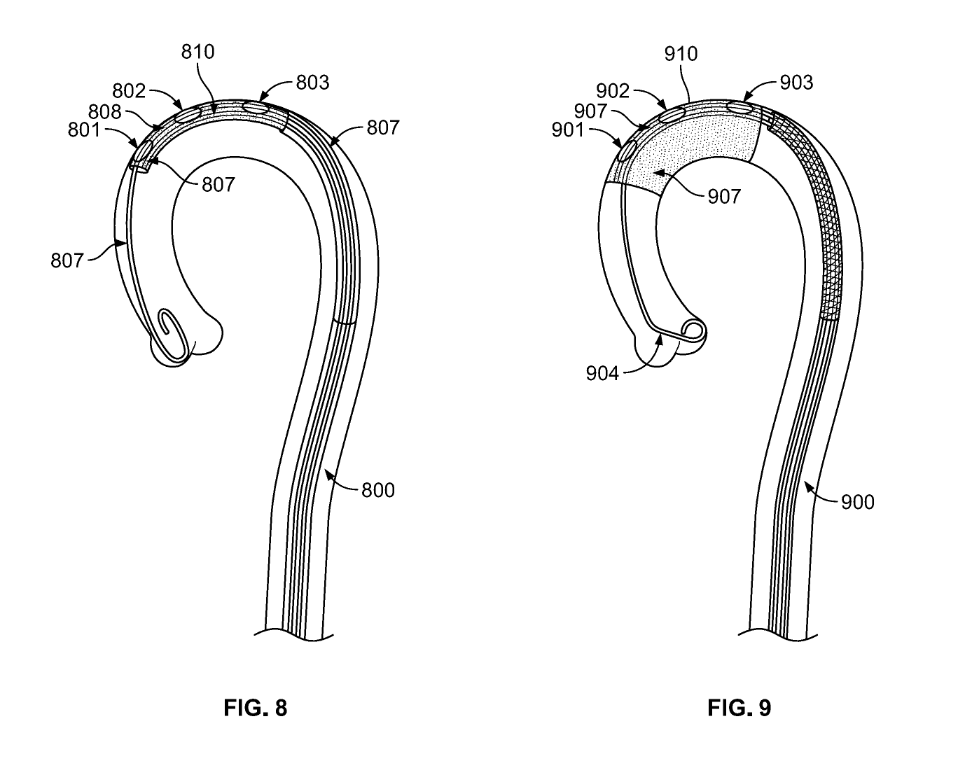

These and other embodiments of the invention are described below with reference to FIGS. 8-12.

FIG. 8 shows a 7 F delivery catheter 800 with a wrapped filter 810 in a folded, undeployed state; the delivery catheter 800 comprises a 0.018″ wire inside the mother lumen and a 0.035″ J wire 807. The delivery filter 800 is shown inside the aorta. The delivery catheter 800 is shown positioned along the right brachiocephalic artery 801; the left common carotid artery 802; and the left subclavian artery 803.

FIG. 9 shows a 7 F delivery catheter 900 with a filter 910 in a deployed state after the delivery catheter 900 has been telescopically retracted and the angled pigtail catheter 904 has been advanced over the 0.035″ J wire, which has been withdrawn. The angled pigtail catheter 904 is jammed/siting in an aortic cusp. The delivery filter 900 is shown inside the aorta. The delivery catheter 900 is shown positioned along the right brachiocephalic artery 901; the left common carotid artery 902; and the left subclavian artery 903.

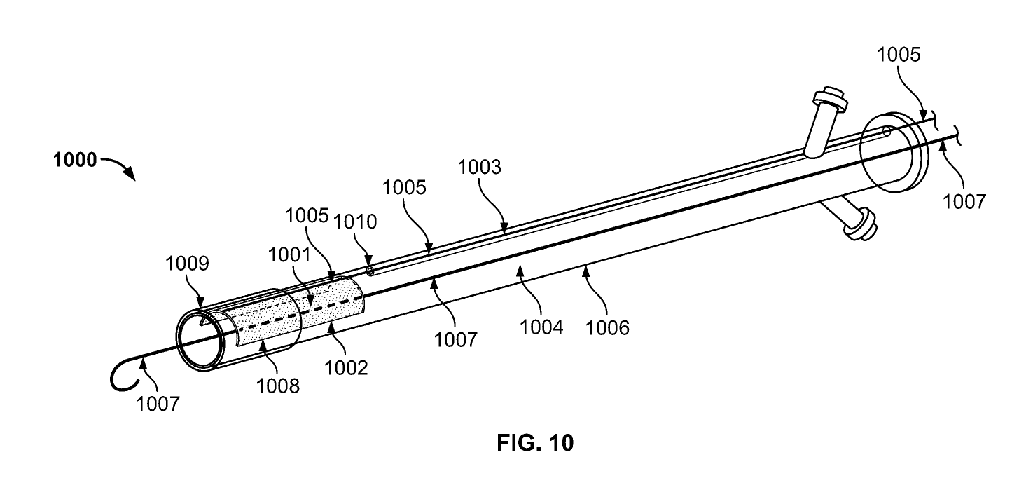

FIG. 10 shows a portion of a kit outside of the box (i.e., in vitro and outside a patient's body) comprising a 7 F delivery catheter 1000 with a wrapped filter 1001 in a folded, undeployed state, inside section A 1002 (12 cm). The delivery catheter 1000 comprises a mother lumen 1004 and a 0.018″ daughter lumen 1003. A 0.018″ wire 1005 is shown inside the mother lumen 1004 of section B 1006 and the single lumen of Section A 1002, attached firmly to the top of the filter 1001 (dorsal). A 0.035″ J wire 1007 is shown inside the mother lumen 1004 of Section B and the single lumen of Section A 1002. Part of the J wire is outside of the front of the delivery catheter 1000. The front most 6 cm 1008 of section A 1002 has extra inbuilt recoil. As can be seen, the 0.018″ wire 1005 and the 0.035″ wire 1007 extend outside of the back of the delivery catheter 1000 and are outside of the patient's blood vessel and body. Two radio opaque markers 1009 and 1010, one placed at the front of section B of the delivery catheter and one embedded along the very same plane at the front end of section A of the delivery catheter, are used to determine the orientation of the daughter lumen, the two markers being placed on the superior aspect of the dual-purpose catheter along the plane of the 0.018″ daughter lumen to facilitate proper alignment and deployment by rotating the delivery catheter to ensure proper filter deployment.

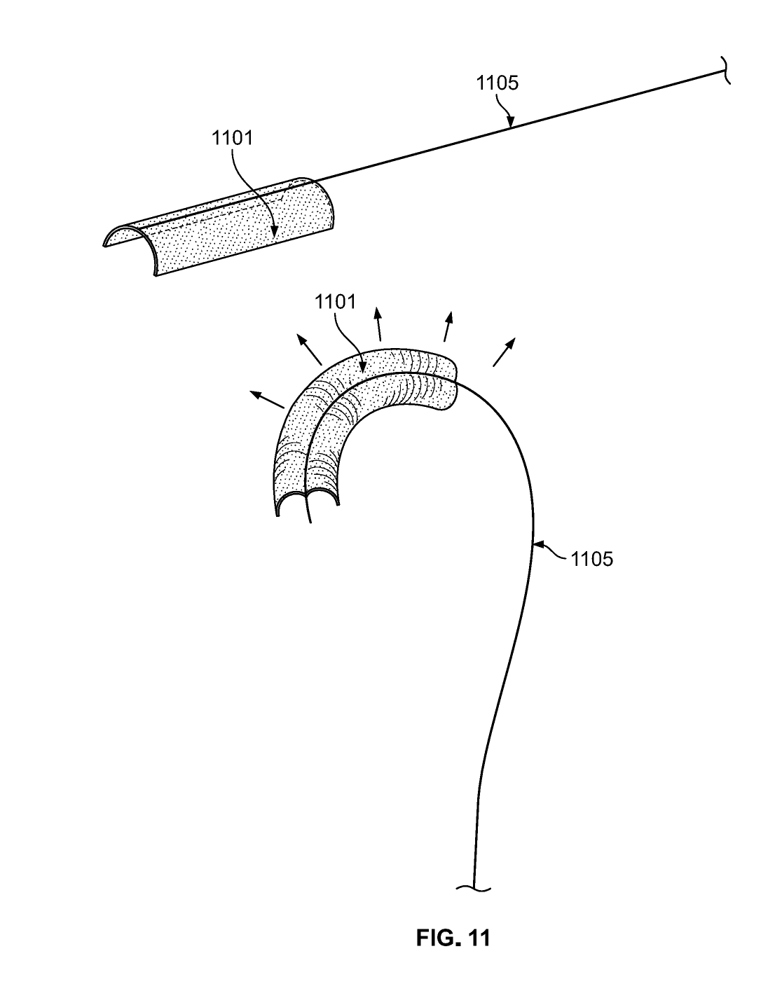

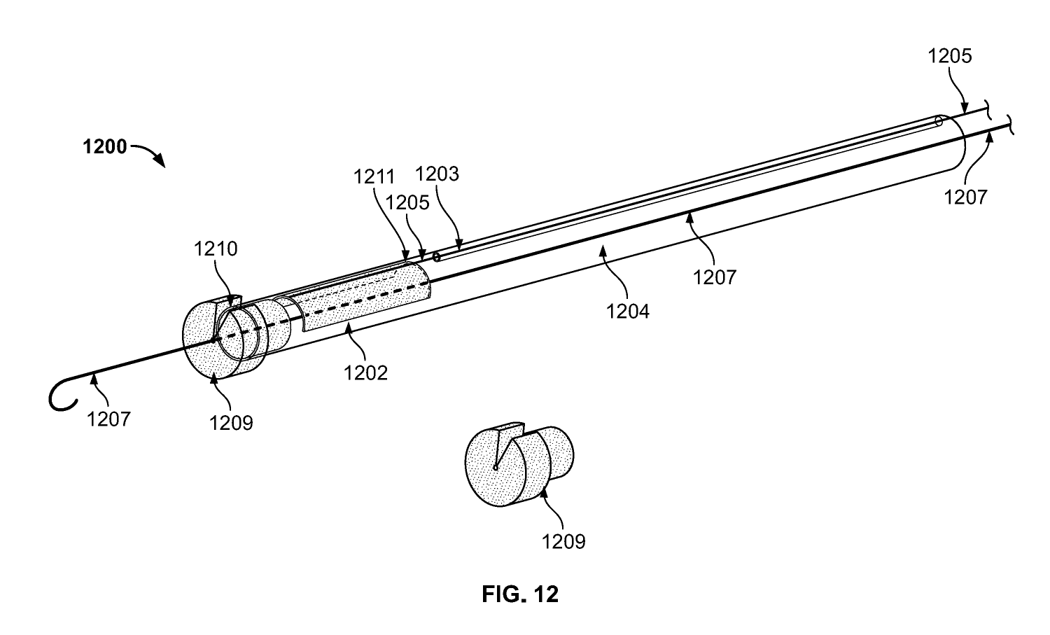

FIG. 11 shows isolated views of the filter 1101 and the 0.018″ wire 1105 (120 cm long with recoil) on top of it. The top view is a view when the delivery catheter is straight, and the bottom view is when the delivery catheter is bent along the greater curvature of the aortic arch, generating centrifugal forces (up arrows) pushing the filter once deployed firmly against the MAAB takeoff.

FIG. 12 shows isolated views of the 0.035″ J wire 1207. Shown also are the daughter lumen 1203, the 0.018″ wire 1205, the mother lumen 1204, and Section A 1202. A port 1209 for the 0.035″ wire is also shown, which has a clefted clipon sponge guarding the front tip of the delivery catheter 1200 to prevent air entry once the catheter is flushed. Two radio opaque markers 1210 and 1211, one placed at the front of section B of the delivery catheter and one embedded along the very same plane at the front end of section A of the delivery catheter, are used to determine the orientation of the daughter lumen, said two markers being placed on the superior aspect of the dual-purpose catheter along the plane of the 0.018 daughter lumen to facilitate proper alignment and deployment by rotating the delivery catheter to ensure proper filter deployment.

### Other Embodiments

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A dual-purpose catheter system for atheroembolic stroke prevention in periprocedure Trans Catheter Aortic Valve replacement (TAVR) or Trans Catheter Mitral Valve Replacement (TMVR) in a patient by preventing atheroembolic debris (embolic plaque) from reaching the patient's brain and for contrast injection and filter immobilization/stabilization, comprising:
a delivery catheter and an angled 5 F single-lumen dedicated pigtail catheter;
(a) the delivery catheter being retractable and 7 French in size and approximately 100 cm long, having a front end adapted to be placed in the aortic arch of the patient and a rear end adapted to hang outside the patient's body when inserted in a patient, and having two sections: a single-lumen Section A that is approximately 12 cm of the front end of said delivery catheter and a two-lumen Section B that is the rest of the delivery catheter; wherein said two lumens of Section B are a mother lumen that is continuous with the single lumen of Section A and a daughter lumen, which is a 0.018″ lumen embedded within the mother lumen along a plane traversing the entire length of Section B;
a rectangular cerebrovascular filter, in a folded, undeployed position inside the single lumen of Section A when the delivery catheter is not retracted or in a unfolded, deployed position when the delivery catheter is retracted, with a strong tensile memory for an outward convexity so as to hug the inner surface of the greater curvature of the aortic arch of the patient; wherein the filter is a thin collapsible mesh 2 to 4 cm wide and approximately 12 cm long, firmly attached to a firm but flexible 0.018″ wire housed in the 0.018″ daughter lumen, running along the entire length of the daughter lumen of Section B and the single lumen of Section A;
said 0.018″ wire is approximately 120 cm in length with the front 12 cm in the single lumen of Section A attached firmly to the dorsum of the filter;
a removable and reinsertable 0.035″ J wire placed in the mother lumen of section B and the single lumen of section A of the delivery catheter; wherein said J wire runs along the entire length of said delivery catheter; wherein said 0.035″ J wire is a moderately stiff approximately 260 cm long wire loosely embedded in the single lumen of section A and the mother lumen of section B of the delivery catheter;
wherein approximately 30 cm of the 0.035″ J wire juts beyond the front end of the delivery catheter and a portion of the 0.035″ J wire hangs outside the back of the delivery catheter;
(b) an angled 5 F single-lumen dedicated pigtail catheter having side holes featuring composite construction adapted to be inserted into the rear end of the delivery catheter and be housed within the single lumen of Section A and the mother lumen of Section B when the filter is in the deployed position;
wherein the delivery catheter is retractable by about 12 cm in the front end, in the retracted position the filter deploys and the angled 5 F pigtail catheter is allowed to be introduced from the rear end of the delivery catheter into the lumen of the mother lumen of section B and the single lumen of section A of the delivery catheter after filter deployment, so as to be advanced over the 0.035″ wire; which is removable and re-insertable;
wherein the front most about 6 cm of the delivery catheter has inbuilt recoil;
wherein said 0.035″ J wire is an introducer and is the wire on which the delivery Catheter is railed into the body for filter and pigtail catheter delivery.

2. The catheter system of claim 1, wherein the delivery catheter is made with composite construction and about 6 cm at the front end of the delivery catheter comprises spring-like flexible ribbing with extra centrifugal recoil and tensile strength.

3. The catheter system of claim 1, wherein two radio opaque markers, a first one placed at the front of section B of the delivery catheter and a second one embedded along the very same plane as the first one at the front of section A of the delivery catheter, are used to determine the orientation of the daughter lumen, said two markers being placed on the superior aspect of the dual-purpose catheter along the plane of the 0.018 daughter lumen to facilitate proper alignment and deployment by rotating the delivery catheter to ensure proper filter deployment.

4. The catheter system of claim 1, wherein the delivery catheter has two proximal flush ports to facilitate air removal.

5. The catheter system of claim 1, wherein the delivery catheter further comprises rubber seals placed at the back end of the delivery catheter, guarding both the 0.018″ and 0.035″ wires against air entry.

6. The catheter system of claim 1, wherein a clip-like removable sponge cap guards the front end of the delivery catheter, allowing saline flush to exit; but once soaked, the sponge cap prevents air from re-entering the catheter by providing a seal against air entry.

7. The catheter system of claim 1, wherein said filter is made of materials that allow for blood to pass through but impedes passage of the larger atheroembolic debris.

8. The catheter system of claim 1, wherein the filter, when deployed, has a strong tensile memory for an outward convexity designed to hug the inner surface of the aortic arch.

9. The catheter system of claim 1, wherein a sponge clip 4 mm in length and 7 F in diameter is wrapped around said 0.035″ wire to cap the front end of the delivery catheter to prevent air entry.

10. A method of preventing or reducing atheroembolic debris from reaching the brain during heart valve repair or replacement in a patient in need of heart valve repair or replacement, comprising:
inserting a delivery catheter of a dual-purpose catheter system of claim 1 into a blood vessel in the leg of the patient and advancing the delivery catheter to the aortic arch of the patient, wherein the filter is not deployed;
Telescopically retracting the delivery catheter by about 12 cm over the 0.018″ wire and the 0.035″ wire, thus deploying the filter;
inserting the angled pigtail catheter into the mother lumen of Section B and the single lumen of section A the pigtail catheter, traversing the mother lumen of section B and the single lumen of section A, and hangs out the front of the delivery catheter underneath the filter; the angled pigtail catheter being used for contrast injection and filter immobilization/stabilization;
removing the 0.035″ J wire from the delivery catheter;
wherein the 0.018″ wire and the 0.035″ J wire extend outside of the back of the delivery catheter and are outside of the patient's blood vessel and body;
performing Trans Catheter Aortic Valve replacement (TAVR) or Trans Catheter Mitral Valve Replacement (TMVR) on the patient while the filter covers the right brachiocephalic artery takeoff, the left common carotid artery takeoff, and the left subclavian artery takeoff snugly to prevent or reduce atheroembolic debris (embolic plaque) from going up the right brachiocephalic artery, the left common carotid artery, and the left subclavian artery to reach the brain.

11. The method according to claim 12, further comprising:
inserting 0.035″ J wire from the rear end of the delivery catheter such that the 0.035 J wire traverses both the single lumen of Section A and the mother lumen of Section B, sitting underneath the pigtail catheter;
withdrawing the pigtail catheter over the 0.035″ J wire and removing the pigtail catheter from the patient's body;
as the pigtail catheter is being withdrawn, simultaneously advancing the 035″ J wire to the aortic root, jamming against an aortic cusp to keep the filter immobilized; holding the dual-purpose delivery catheter and the 0.035″ wire firmly in one hand, withdrawing the 0.018″ wire gently until the filter attached to it is fully re-encased in the single lumen section A of the dual-purpose delivery catheter;
holding the 0.018″ wire and the dual-purpose catheter firmly in one hand and pinning the 0.035″ J wire down to a surface with the other hand to keep it immobilized;
removing the dual-purpose catheter housing the filter out of the body of the patient over the 0.035″ J wire; and
removing the 0.035″ wire from the body of the patient.

12. A kit comprising a catheter system of claim 1, comprising a delivery catheter and an angled 5 F single-lumen dedicated pigtail catheter, for use on a patient during a cardiac intervention selected from Trans Catheter Aortic Valve replacement (TAVR) or Trans Catheter Mitral Valve Replacement (TMVR) to prevent atheroembolic debris (embolic plaque) from reaching the patient's brain and for contrast injection and filter immobilization/stabilization.

13. A delivery catheter that is part of a dual-purpose catheter system for use on a patient during a cardiac intervention selected from Trans Catheter Aortic Valve replacement (TAVR) or Trans Catheter Mitral Valve Replacement (TMVR);
the delivery catheter being retractable and 7 French in size and approximately 100 cm long, having a front end adapted to be placed in the aortic arch of the patient and a rear end adapted to hang outside the patient's body when inserted in a patient, and having two sections: a single-lumen Section A that is approximately 12 cm of the front end of said delivery catheter and a two-lumen Section B that is the rest of the delivery catheter; wherein said two lumens of Section B are a mother lumen that is continuous with the single lumen of Section A and a daughter lumen, which is a 0.018″ lumen embedded within the mother lumen along a plane traversing the entire length of Section B;
a rectangular cerebrovascular filter, in a folded, undeployed position inside the single lumen of Section A when the delivery catheter is not retracted or in a unfolded, deployed position when the delivery catheter is retracted, with a strong tensile memory for an outward convexity so as to hug the inner surface of the greater curvature of the aortic arch of the patient; wherein the filter is a thin collapsible mesh 2 to 4 cm wide and approximately 12 cm long, firmly attached to a firm but flexible 0.018″ wire housed in the 0.018″ daughter lumen, running along the entire length of the daughter lumen of Section B and the single lumen of Section A;
said 0.018″ wire is approximately 120 cm in length with the front 12 cm in the single lumen of Section A attached firmly to the dorsum of the filter;
a removable and reinsertable 0.035″ J wire placed in the mother lumen of section B and the single lumen of section A of the delivery catheter; wherein said J wire runs along the entire length of said delivery catheter; wherein said 0.035″ J wire is a moderately stiff approximately 260 cm long wire loosely embedded in the single lumen of section A and the mother lumen of section B of the delivery catheter;
wherein approximately 30 cm of the 0.035″ J wire juts beyond the front end of the delivery catheter and a portion of the 0.035″ J wire hangs outside the back of the delivery catheter.
